(19) United States
(12) Patent Application Publication
(10) Pub. No.: US 2006/0058310 A1
(43) Pub. Date: Mar. 16, 2006

(54) Remedies for vertebral canal stenosis

(76) Inventors: Yoshifumi Takenobu, Mishima-gun (JP); Yoshihisa Kamanaka, Mishima-gun (JP); Takaaki Obata, Mishima-gun (JP); Hidenori Ito, Mishima-gun (JP)

Correspondence Address: SUGHRUE MION, PLLC, 2100 PENNSYLVANIA AVENUE, N.W., SUITE 800, WASHINGTON, DC, 20037, US

(21) Appl. No.: 10/534,051

(22) PCT Filed: Nov. 13, 2003
(86) PCT No.: PCT/JP03/14454

§ 371 Date: May 5, 2005

(30) Foreign Application Priority Data
Nov. 14, 2002 (JP) 2002-330425

(51) Int. Cl. A61K 31/517; A61K 31/498; A61K 31/4747; A61K 31/426; A61K 31/557

(52) U.S. Cl. 514/250; 514/369; 514/266.3; 514/278; 514/573

(57) ABSTRACT

The present invention relates to a preventive and/or therapeutic agent for spinal canal stenosis comprising aldose reductase inhibitory compounds. A representative example of aldose reductase inhibitory compounds is the compound represented by formula (I): 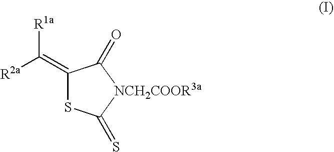
wherein all the symbols represent the same meaning as described in the specification; a salt of its acid when R^{3a} represents hydrogen, or solvate thereof The therapeutic agent of the present invention is effective for prevention and/or therapy for spinal canal stenosis and the like, such as lumbar spinal canal stenosis.

### TECHNICAL FIELD

The present invention relates to a therapeutic agent for spinal canal stenosis. More specifically, the present invention relates to a preventive and/or therapeutic agent for spinal canal stenosis comprising an aldose reductase inhibitory compound.

### BACKGROUND ART

The internal space enclosed with each vertebral body and processus spinalis from the cervical vertebra to the sacral vertebra is called spinal canal. The spinal canal stenosis shows various symptoms by narrowing spinal canal due to the hypertrophic degeneration of the spine, one of constituents of spinal canal, and the yellow ligament and due to the projection of the intervertebral disc etc., followed by compressing the nerve tissue of the nerve root and the cauda equina, etc. The spinal canal stenosis is classified into the cervical spinal canal stenosis, the thoracic spinal canal stenosis, the lumbar spinal canal stenosis and the wide spinal canal stenosis by narrow parts of spinal canal. Their symptoms by the nerve compression are lumber pain, upper or lower limbs pain, and numbness, etc. Especially, when the cauda equina nerve is injured, lumber pain, lower limbs pain, numbness and languidness deteriorate. This symptom is called an intermittent claudication.

On the other hand, the aldose reductase is an enzyme that reduces aldose in the body, for example, glucose and galactose, to the corresponding polyol, for example, sorbitol and garactitol. The sorbitol and garactitol generated by a hyperactivation of this enzyme under the excessive level of glucose concentration in diabetic patients and the like are accumulated in the crystalline lens, the peripheral nerve and the kidney, etc. And as a result, the complications of the retinopathy, the diabetic cataract, the peripheral neuropathy and the renal damage, etc. are caused. The aldose reductase inhibitors are known to be effective for the treatment and the prevention of complications of chronic diabetes mellitus by inhibiting the aldose reductase.

As aldose reductase inhibitors, for example, the following compounds are known.

A compound represented by formula (I):

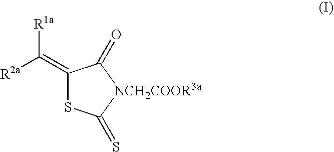

wherein all groups are the same meaning as described hereinafter; is reported to have inhibitory activity of aldose reductase and be effective for a prevention and/or therapy of chronic diabetic complication, such as cardiovascular disease, nephropathy, retinopathy, diabetic cataract and neuropathy, and infection complication derived from aldose reductase, such as neuralgic neuropathy, retinopathy, diabetic cataract and tubular renal disease-like nephropathy. (U.S. Pat. No. 4,464,382 and U.S. Pat. No. 4,831,045).

A compound represented by formula (II):

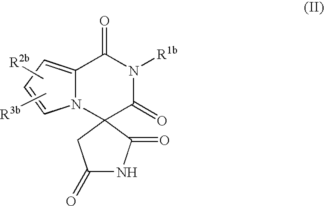

wherein all groups are the same meaning as described hereinafter; is reported to have inhibitory activity of aldose reductase and be effective for a prevention and/or therapy of diabetic complication, such as cataract, retinopathy, keratopathy, neuropathy and nephropathy (JP-A-5-186472 and JP-A-5-345784).

A compound represented by formula (III):

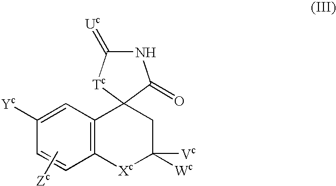

wherein all groups are the same meaning as described hereinafter; is reported to have inhibitory activity of aldose reductase and be effective for a prevention and/or therapy of various diabetic complication, such as diabetic cataract, diabetic neuropathy, diabetic retinopathy and diabetic nephropathy.

A compound represented by formula (IV):

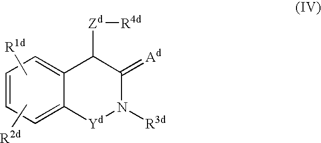

wherein all groups are the same meaning as described hereinafter; is reported to have inhibitory activity of aldose reductase and be effective for a prevention and/or therapy of diabetic complication (U.S. Pat. No. 4,734,419 and U.S. Pat. No. 4,883,800).

However, it has never reported that compounds of aldose reductase inhibitor are useful for spinal canal stenosis.

### DISCLOSURE OF THE INVENTION

Therapy of spinal canal stenosis is surgery in severe case, and basically conservative therapy, for example pharmacotherapy, exercise therapy for strengthening such as back and abdominal muscles, thermotherapy therapy such as hot pack, acupuncture therapy for relieving pain, orthotic therapy such as corset and the like in mild case. However, there is no therapy for various symptoms of spinal canal stenosis to improve satisfactorily.

Many of spinal canal stenosis patients are treated with conservative therapy and many of them improve their condition by combination of conservative therapies. However, oral prostaglandin E1 derivatives for the improvement of the circulation in the nerve tissue, are the only launched drug for spinal canal stenosis. Therefore, the present inventors have made extensive studies to find a therapeutic agent for spinal canal stenosis, and as a result, have found that an aldose reductase inhibitor is unexpectedly able to improve the condition of spinal canal stenosis, and then have completed the present invention. It has not been reported that the aldose reductase inhibitors are effective for the treatment of spinal canal stenosis. These inventors confirmed for the first time that the aldose reductase inhibitors were effective for the treatment of the spinal canal stenosis by using a rat of gait disturbance model by cauda equina compression (J. Neurosci. Methods, 104(2), 191-198 (2002)) known as a model with the spinal canal stenosis.

Namely, the present invention relates to the followings:

1. a preventive and/or therapeutic agent for spinal canal stenosis, which comprises an aldose reductase inhibitory compound,

2. the preventive and/or therapeutic agent for spinal canal stenosis according to the above 1, wherein the spinal canal stenosis is cervical spinal canal stenosis, thoracic spinal canal stenosis, lumbar spinal canal stenosis or wide spinal canal stenosis,

3. the preventive and/or therapeutic agent for spinal canal stenosis according to the above 1, wherein the agent is used for improving paralysis, hypoesthesia, pain or numbness,

4. the preventive and/or therapeutic agent for spinal canal stenosis according to the above 1, wherein the agent is used for improving physical ability,

5. the preventive and/or therapeutic agent for spinal canal stenosis according to the above 4, wherein the agent for improving physical ability is used for improving reduction of muscle power, intermittent claudication and gait disability,

6. the preventive and/or therapeutic agent for spinal canal stenosis according to the above 1, wherein the agent is used for improving dysuria or dyschezia,

7. the preventive and/or therapeutic agent for spinal canal stenosis according to the above 1, wherein the aldose reductase inhibitory compound is represented by formula (I) wherein all groups are the same meaning as described hereinafter, a salt when R^{3a} is hydrogen, or a solvate thereof,

8. the preventive and/or therapeutic agent for spinal canal stenosis according to the above 1, wherein the aldose reductase inhibitory compound is a compound represented by formula (II), wherein the definition of each group is the same meaning as hereinafter, a salt thereof or a solvate thereof, a compound represented by formula (III), wherein the definition of each group is the same meaning as hereinafter; a salt thereof or a solvate thereof, or a compound represented by formula (IV), wherein definition of each group is the same meaning as hereinafter, a salt thereof or a solvate thereof,

9. the preventive and/or therapeutic agent for spinal canal stenosis according to the above 7, wherein the aldose reductase inhibitory compound is 5-[(1Z,2E)-2-methylphenylpropenylidene]-4-oxo-2-thioxo-3-thiazolidine acetic acid,

10. the preventive and/or therapeutic agent for spinal canal stenosis according to the above 8, wherein the aldose reductase inhibitory compound is (R)-2-(4-bromo-2-fluorobenzyl)spiro[1,2,3,4-tetrahydropyrrolo[1,2-a]pyrazine-4,3′-pyrrolidine]-1,2′,3,5′-tetrone, (2S,4S)-6-fluoro-2′,5′-dioxospiro[3,4-dihydro-2H-1-benzopyran-4,4′-imidazolidine]-2-carboxamide or 2-[3-(4-bromo-2-fluorobenzyl)-7-chrolo-2,4-dioxo-1,2,3,4-tetrahydroquinazolin-1-yl]acetic acid,

11. the preventive and/or therapeutic agent for spinal canal stenosis according to the above 1, wherein an aldose reductase inhibitory compound is

(1) DL-spiro(2-fluoro-9H-fluoren-9,4′-imidazolidine)-2′,5′-dione,

(2) 2,7-difluoro-4,5-dimethoxyspiro[9H-fluoren-9,4′-imidazolidine]-2′,5′-dione,

(3) N-[3,5-dimethyl-4-(nitromethylsulphonyl)phenyl]-2-(2-methylphenyl)acetamide,

(4) N-(carboxymethyl)-7-fluoro-N-methyl-9-oxoxanthin-2-sulphoamide,

(5) 3-(4-methoxy-5-oxo-3-phenyl-2,5-dihydrofuran-2-yl)propanoic acid ethyl ester,

(6) 2-formamide-3-[5′-(2-formamide-1-hydroxyethyl)-2,2′-dihydroxybiphenyl-5-yl]-3-hydroxypropyonic acid,

(7) 2-[3-methyl-5-(4,5,7-trifluorobenzothiazol-2-ylmethyl)phenyl]acetic acid,

(8) 2-[5-fluoro-2-[N-(3-nitrobenzyl)thiocarbamoyl]phenoxyacetic acid,

(9) 8′-chrolo-2′,3′-dihydrospiro[pyrrolidine-(3,6′)(5′H)-pyrro-[1,2,3-de][1,4]benzoxazine]-2,5,5′-trione,

(10) 2-[1-(3,4-dichrolobenzyl)-2,4-dioxo-1,2,3,4-tetrahydroquinazolin-3-yl]acetic acid,

(11) 2-[4-(4,5,7-trifluoro-benzothiazol-2-yl)methyl-3-oxo-3,4-dihydro-2H-1,4-benzothiazin-2-yl]acetic acid,

(12) 1-(benzo[b]thiophen-2-ylsulphonyl)hydantoin,

(13) 1-(3-bromobenzo[b]furan-2-ylsulphonyl)hydanthione,

(14) 3-(carboxymethyl)-1-(3-nitrobenzyl)parabanic acid,

(15) 1′,3′-bis(acetoxymethyl)spiro[fluoren-9,4′-imidazolidine]-2′,5′-dione,

(16) 2,8-diisopropyl-3-thioxo-3,4-dihydro-2H-1,4-benzoxazine-4-acetic acid,

(17) N-[5-(trifluoromethyl)-6-methoxy-1-naphthalenyl]thioxomethyl]-N-methylglycine),

(18) (2,6-dimethylphenylsulphonyl)nitromethane,

(19) N-[4-(2,4-dioxothiazolidin-5-ylmethyl)phenyl]-1-phenyl-cyclopropane-1-carboxamide,

(20) 2-[3-oxo-4-(4,5,7-trifluorobenzothiazol-2-ylmethyl)-3,4-dihydro-2H-1,4-benzothiazin-2-yl]acetic acid,

(21) 2-[3,7-dimethylocta-2(E),6-dienyl]-2,3-epoxy-5,7-dihydroxy-6-methyl-1,2,3,4-tetrahydronaphthalene-1,4-dione, (22) 6-fluoro-2-methylspiro[chroman-4,4′-imidazolidine]-2′,5′-dione,

(23) (S)-6-fluorospiro(chroman-4,4′-imidazolidine)-2′,5′-dione,

(24) 3,4-dihydro-4-oxo-3-[[5-(trifluoromethyl)-2-benzothiazolyl]methyl]-1-phthalazine acetic acid,

(25) 5-(3-ethoxy-4-pentyloxyphenyl)-2,4-thiazolidinedione,

(26) 3-[(4-bromo-2-fluorophenyl)methyl]-3,4-dihydro-4-oxo-1-phthalazine acetic acid,

(27) ascorbyl gamolenate,

(28) ICI-10552,

(29) ICI-215918,

(30) JTT-811,

(31) lindolrestat,

(32) salfredins,

(33) TJN-732,

(34) TAT,

(35) thiazocin-A,

(36) axillarin, or

(37) minalrestat,

12. a medicine which comprises aldose reductase inhibitory compounds according to the above 1 in combination with at least one pharmaceutical agent selected from aldose reductase inhibitory compounds according to the above 1, prostaglandins, prostaglandin derivatives formulations, nonsteroidal anti-inflammatory drugs, vitamin compounds, muscle relaxants, antidepressants, poly ADP-ribose polymerase inhibitors, excitatory amino acid receptor antagonists, radical scavengers, astrocyte modulators, IL-8 receptor antagonists, and immunosuppressive drugs,

13. a method for prevention and/or treatment for spinal canal stenosis, which comprises administering to a mammal an effective amount of an aldose reductase inhibitory compound, and

14. use of an aldose reductase inhibitory compound for preparation of a preventive and/or therapeutic agent for spinal canal stenosis.

The aldose reductase inhibitory compounds of the present invention include all of the agents which have aldose inhibitory activity. In addition, the aldose reductase inhibitory compounds not only which has been found up to now but also which will be found in future are included.

The aldose reductase inhibitory compounds include epalrestat (U.S. Pat. No. 4,464,382), fidarestat (SNK-860; U.S. Pat. No. 4,740,517), (R)-2-(4-bromo-2-fluorobenzyl)spiro[1,2,3,4-tetrahydropyrrolo[1,2-a]pyrazine-4,3′-pyrrolidine]-1,2′,3,5′-tetrone (AS-3201; JP-A-5-186472), zenarestat (FK-366; U.S. Pat. No. 4,734,419 and U.S. Pat. No. 4,883,800), DL-spiro(2-fluoro-9H-fluoren-9,4′-imidazolidine)-2′,5′-dione (imirestat, AL-1567; JP-A-60-89469), 2,7-difluoro-4,5-dimethoxyspiro[9H-fluoren-9,4′-imidazolidine]-2′,5′-dione (AL-4114; JP-A-60-89469), N-[3,5-dimethyl-4-(nitromethylsulfonyl)phenyl]-2-(2-methylphenyl)acetamide (ZD-5522), N-(carboxymethyl)-7-fluoro-N-methyl-9-oxoxanthine-2-sulfoamide (BAL-ARI8; EP307879), 3-(4-methoxy-5-oxo-3-phenyl-2,5-dihydrofuran-2-yl)-propanoic acid ethyl ester (FR-62765; EP189272), 2-formamide-3-[5′-(2-formamide-1-hydroxyethyl)-2,2′-dihydrooxybiphenyl-5-yl]-3-hydroxypropionic acid (WF-2421(FR-90028); JP-A-2-72144), 2-[3-methyl-5-(4,5,7-trifluorobenzothiazol-2-ylmethyl)-phenyl]acetic acid (GP-1447; EP714893), 2-[5-fluoro-2-[N-(3-nitrobenzyl)thiocarbamoyl]phenoxy]acetic acid (IDD-598), 8′-chrolo-2′,3′-dihydrospiro-[pyrrolidine-(3,6′)(5′H)-pyrrolo[1.2.3-de][1,4]benzoxazine]-2,5,5′-trione (ADN-138), 2-[1-(3,4-dichrolobenzyl)-2,4-dioxo-1,2,3,4-tetrahydroquinazolin-3-yl]acetic acid (ADN-311), 2-[4-(4,5,7-trifluorobenzo-thiazol-2-yl)methyl-3-oxo-3,4-dihydro-2H-1,4-benzothiazin-2-yl]acetic acid (SG-210), 1-(benzo[b]thiophen-2-ylsulfonyl)hydantoin (M-16049; EP355827)), 1-(3-bromobenzo[b]furan-2-ylsulfonyl)hydanthion (M-16209; EP355827), 3-(carboxymethyl)-1-(3-nitrobenzyl)parabanic acid (NZ-314; EP353198), 1′,3′-bis(acetoxymethyl)spiro[fluoren-9,4′-imidazolidine]-2′,5′-dione (CP-AR-3192; U.S. Pat. No. 4,853,401), 2,8-diisopropyl-3-thioxo-3,4-dihydro-2H-1,4-benzoxazine-4-acetic acid (AD-5467; EP243018), N-[5-(trifluoromethyl)-6-methoxy-1-naphthalenyl]thioxomethyl]-N-methylglycine (tolrestat; U.S. Pat. No. 4,439,617), (2,6-dimethylphenylsulfonyl)nitromethane (ICI-215918), N-[4-(2,4-dioxothiazolidin-5-ylmethyl)phenyl]-1-phenyl-cyclopropane-1-carboxyamide (DN-108; WO97/32863), 2-[3-oxo-4-(4,5,7-trifluorobenzothiazol-2-ylmethyl)-3,4-dihydro-2H-1,4-benzothiazin-2-yl]acetic acid (SPR-210; EP492667), 2-[3,7-dimethylocta-2(E),6-dienyl]-2,3-epoxy-5,7-dihydroxy-6-methyl-1,2,3,4-tetrahydronaphthalene-1,4-dione (A-74863a; JP-A-7-10857), ICI-10552, ICI-215918, 6-fluoro-2-methylspiro[chroman-4,4′-imidazolidine]-2′,5′-dione (methosorbinil; JTT-811), lindolrestat (IDD-676; WO99/50268), (S)-6-fluorospiro(chroman-4,4′-imidazolidine)-2′,5′-dione (sorbinil; U.S. Pat. No. 4,474,967), 3,4-dihydro-4-oxo-3-[[5-((trifluoromethyl)-2-benzothiazolyl]methyl]-1-phthalazine acetic acid (zopolrestat; EP222576), ascorbyl gamolenate (SC-103, CA2143603), 5-(3-ethoxy-4-(pentyloxyphenyl)-2,4-thiazolidinedione (risarestat, CT-112; EP33617), salfredins (TJN-732, TAT(EP421365)), thiazocin-A, axillarin, 3-[(4-bromo-2-fluorophenyl)methyl]-3,4-dihydro-4-oxo-1-phthalazine acetic acid (ponalrestat; EP2895), minalrestat (WAY-121509; EP365324) and the like.

In addition, the compound with aldose reductase inhibitory activity, described in the specification of U.S. Pat. No. 4,464,382, U.S. Pat. No. 4,740,517, JP-A-5-186472, JP-A-5-345784, U.S. Pat. No. 4,734,419, U.S. Pat. No. 4,883,800, JP-A-60-89469, EP307879, EP189272, JP-A-2-72144, EP714893, WO99/50268, EP355827, EP355827, EP353198, U.S. Pat. No. 4,474,967, EP222576, U.S. Pat. No. 4,853,401, CA2143603, EP33617, EP243018, EP421365, U.S. Pat. No. 4,439,617, EP2895, EP365324, WO97/32863, EP492667, JP-A-7-10857, EP1236720, EP1236720, WO92/17446, JP-A-2002-241347, EP269355, JP-A-62-67075, EP252713, EP305947, EP322255, WO98/28265, EP17379, WO89/09773 and the like are useful. Therefore, the present invention includes the application for these compounds to use of the present invention.

Among the compounds of the present invention, a preferred compound includes the compound represented by formula (I):

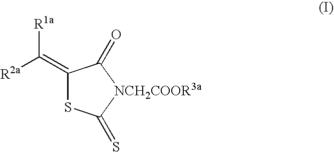

wherein

1) R^{1a} and R^{2a} are the same or different and each represents phenyl which may be substituted by at least one group selected from the following (1)-(10):

(1) halogen,

(2) trifluoromethyl,

(3) hydroxyl,

(4) nitro,

(5) carboxyl,

(6) amino which may be substituted by C1-4 alkyl,

(7) C1-4 alkyl, alkoxy or alkylthio,

(8) phenyl,

(9) a heterocyclic group containing at least one atom selected from a nitrogen atom, a sulfur atom and an oxygen atom, which may be substituted by at least one group selected from (a) halogen, (b) trifluoromethyl, (c) phenyl, (d) nitro, (e) hydroxyl, (f) carboxyl, (g) amino which may be substituted by C1-4 alkyl, (h) C1-4 alkyl, (j) C1-4 alkoxy, and (k) C1-4 alkylthio, or

(10) C1-4 alkyl which may be substituted by at least one substituent selected from hydroxyl, phenyl, and a heterocyclic group selected from described above (9),

2) R^{1a} is hydrogen and R^{2a} is the following (1)-(6):

(1) C4-7 cycloalkyl or cycloalkenyl which may be substituted by at least one C1-4 alkyl,

(2) anthryl or naphthyl,

(3) phenyl which may be substituted by at least one group selected from the following (a)-(k):
(a) halogen,
(b) trifluoromethyl,
(c) hydroxyl,
(d) nitro,
(e) carboxyl,
(f) amino which may be substituted by C1-4 alkyl
(g) C1-4 alkyl, alkoxy or alkylthio,
(h) phenyl,
(j) a heterocyclic group containing at least one atom selected from a nitrogen atom, a sulfur atom and an oxygen atom, which may be substituted by at least one group selected from (i) halogen, (ii) trifluoromethyl, (iii) phenyl, (iv) nitro, (v) hydroxyl, (vi) carboxyl, (vii) amino which may be substituted by C1-4 alkyl, (viii) C1-4 alkyl, (ix) C1-4 alkoxy, and (x) C1-4 alkylthio,
(k) C1-4 alkyl which is substituted by at least one group selected from hydroxyl, phenyl, and the heterocyclic group described above (j),

(4) a heterocyclic group containing at least one atom selected from a nitrogen atom, a sulfur atom and an oxygen atom, which may be substituted by at least one group selected from the following (a)-(k):
(a) halogen,
(b) trifluoromethyl,
(c) phenyl,
(d) nitro,
(e) hydroxyl,
(f) carboxyl,
(g) amino which may be substituted by C1-4 alkyl,
(h) C1-4 alkyl, alkoxy or alkylthio,
(j) oxo, or
(k) C1-4 alkyl substituted by at least one substituent selected from hydroxyl, phenyl, and the heterocyclic group described above (j) in (3),

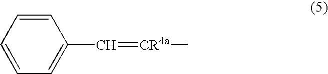

wherein R^{4a} is hydrogen or C1-4 alkyl, or

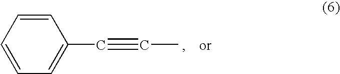

3) R^{1a} taken together with R^{2a} is tetramethylene or pentamethlene;

R^{3a} is

(1) hydrogen,

(2) C1-12 alkyl,

(3) C7-13 aralkyl,

(4) C4-7 cycloalkyl or cycloalkenyl which may be substituted by at least one C1-4 alkyl,

(5) phenyl which may be substituted by at least one group selected from the following (a)-(k):
(a) halogen,
(b) trifluoromethyl,
(c) hydroxyl,
(d) nitro,
(e) carboxyl,
(f) amino which may be substituted by C1-4 alkyl,
(g) alkoxy or alkythio which may be substituted by C1-4 alkyl,
(h) phenyl,
(j) a heterocyclic group containing at least one atom selected from a nitrogen atom, a sulfur atom and an oxygen atom, which may be substituted by at least one group selected from (i) halogen, (ii) trifluoromethyl, (iii) phenyl, (iv) nitro, (v) hydroxyl, (vi) carboxyl, (vii) amino which may be substituted by C1-4 alkyl, (viii) C1-4 alkyl, (ix) C1-4 alkoxy, and (x) C1-4 alkylthio, and,
(k) C1-4 alkyl substituted by at least one substituent selected from hydroxyl, phenyl, and the heterocyclic group described above (j), or a salt of acid thereof when R^{3a} represents hydrogen, or a solvate thereof In the definition of formula (I),

C1-4 alkyl is methyl, ethyl, propyl, butyl, or isomers thereof,

C1-4 alkoxy is methoxy, ethoxy, propoxy, butoxy or isomers thereof,

C1-4 alkylthio is methylthio, ethylthio, propylthio, butylthio, or isomers thereof,

C4-7 cycloalkyl is cyclobutyl, cyclopentyl, cyclohexyl, cycloheptyl, or isomers thereof,

C4-7 cycloalkenyl is cyclobutenyl, cyclopentenyl, cyclohexenyl, cycloheptenyl or isomers thereof, and

C1-12 alkyl is methyl, ethyl, propyl, butyl, pentyl, hexyl, heptyl, octyl, nonyl, decyl, undecyl, dodecyl or isomers thereof

In addition, a preferred compound includes a compound represented by formula (II):

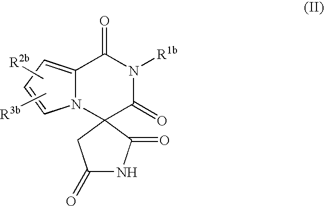

wherein R^{1b} represents (1) hydrogen, (2) lower alkyl, (3) substituted or unsubstituted aryl(lower alkyl), (4) substituted or unsubstituted aryl, or (5)

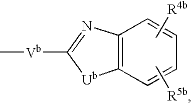

wherein R^{4b} and R^{5b} are the same or different and each represents (a) hydrogen, (b) halogen, (c) trifluoromethyl, (d) lower alkyl, (e) lower alkoxy, (f) acyl, (g) nitro, (h) amino, (i) lower alkylamino, or (j) di(lower alkyl)amino; U^{b} represents (a) oxygen, (b) sulfur, or (c) —NR^{6b}—wherein NR^{6b} represents hydrogen or lower alkyl, and V^{b} represents lower alkyl;

wherein R^{2b} and R^{3b} are the same or different and each represents (1) hydrogen, (2) halogen, (3) lower alkyl, (4) lower alkoxy, (5) acyl, (6) nitro, (7) amino, (8) lower alkylamino, (9) di(lower alkyl)amino, (10) allyl or (11) allyl which is substituted by lower alkyl, lower alkoxy or acyl, or

a salt thereof, or a solvate thereof,

a compound represented by formula (III):

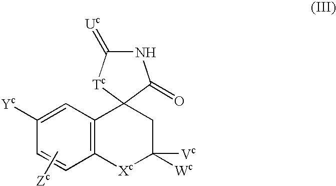

wherein T^{c} represents sulfur or NH;

U^{c} represents oxygen, sulfur or imino;

one of V^{c} and W^{c} represents hydrogen; halogenomethyl; 1H-tetrozol-5-yl; —COOR^{c} wherein R^{c} is hydrogen, alkyl, —(CH₂CH₂O)ₙCH₃ wherein n is an integer of 1 to 113, or substituted phenyl; 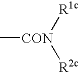
wherein R^{1c} and R^{2c} are the same or different and each represents hydrogen, alkyl, —(CH₂CH₂O)ₙCH₃ wherein n is an integer of 1 to 113, or substituted phenyl; —CH₂OR^{3c} wherein R^{3c} is hydrogen or alkyl; or 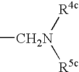
wherein R^{4c} and R^{5c} are the same or different and each represents hydrogen or alkyl, and the other represents hydrogen or alkyl;

X^{c} represents oxygen or sulfur;

Y^{c} and Z^{c} are the same or different and each represents hydrogen, halogen, alkyl, alkoxy, or alkylthio, or

a salt thereof or a solvate thereof, and

a quinazoline derivative represented by formula (IV):

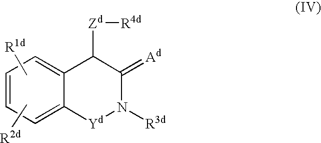

wherein R^{1d} and R^{2d} are the same or different and each represents hydrogen, halogen, lower alkoxy, or halo(lower alkyl);

R^{3d} represents (1) aryl or aryl(lower alkyl) which may be substituted, or (2) heterocyclic ring-(lower alkyl);

R^{4d} represents carboxy or protected carboxy;

A^{d} represents oxygen or sulfur;

Y^{d} represents carbonyl, thiocarbonyl, or sulfonyl;

Z^{d} represents lower alkylene, or

a salt thereof or a solvate thereof

According to the present invention, unless otherwise indicated and as is apparent for those skilled in the art, symbol [symbol] indicates that it is bound to the opposite side of the sheet (namely α-configuration), symbol [symbol] indicates that it is bound to the front side of the sheet (namely β-configuration), symbol [symbol] indicates that it is α-, β- or a mixture thereof, symbol [symbol] indicates that it is a mixture of α-configuration and β-configuration, symbol [symbol] indicates a single bond or double bond, symbol [symbol] indicates a double bond or triple bond, and symbol [symbol] indicates a single bond, double bond, or triple bond.

Furthermore, among the compounds of the present invention, a more preferred compound is

5-[(1Z,2E)-2-methylphenylpropenylidene]-4-oxo-2-thioxo-3-thiazolidine acetic acid (epalrestat) included in formula (I),

(R)-2-(4-bromo-2-fluorobenzyl)spiro[1,2,3,4-tetrahydropyrrolo[1,2-a]pyrazine-4,3′-pyrrolidin]-1,2′,3,5′-tetrone (AS-3201) included in formula (II),

(2S,4S)-6-fluoro-2′,5′-diioxospiro[3,4-dihydro-2H-1-benzopyran-4,4′-imidazolidine-2-carboxamide (SNK-860; fidarestat) included in formula (III), or

2-[3-(4-bromo-2-fluorobenzyl)-7-chrolo-2,4-dioxo-1,2,3,4-tetrahydroquinazolin-1-yl]acetic acid (FK-366; zenarestat) included in formula (IV).

The compound of the present invention may be converted into salts by a known method.

In the specification, salts include salts of alkali metals, salts of alkaline earth metals, ammonium salts, salts with organic amines, acid addition salts and the like.

The salts are preferably non-toxic and water-soluble. Suitable salts include, for example, salts of alkali metals (potassium or sodium, etc.), salts of alkaline earth metals (calcium or magnesium, etc.), ammonium salts, pharmacologically acceptable salts with organic amines (tetramethylammonium, triethylamine, methylamine, dimethylamine, cyclopentylamine, benzylamine, phenethylamine, piperidine, monoethanolamine, diethanolamine, tris(hydroxymethyl)methylamine, lysine, arginine or N-methyl-D-glucamine, etc.).

The acid addition salts are preferably non-toxic and water-soluble. Suitable acid addition salts include salts of inorganic acids (hydrochloride, hydrobromide, hydroiodide, sulfate, phosphate, nitrate, etc.), salts of organic acids (acetate, trifluoroacetate, lactate, tartrate, oxalate, fumarate, maleate, benzoate, citrate, methanesulfonate, ethanesulfonate, benzenesulfonate, toluenesulfonate, isethionate, glucuronate, gluconate, etc.), and the like.

The compound of the present invention and salts thereof may be converted into non-toxic and pharmaceutically acceptable solvates by a known method.

The solvates are preferably non-toxic and water-soluble. Suitable solvates include water, alcohol solvents (ethanol, etc.), and the like.

Unless otherwise specified, the present invention includes all isomers. For example, alkyl, alkenyl, alkynyl, alkoxy, alkylthio, alkylene, alkenylene, alkynylene, etc. include straight or branched ones. In addition, the present invention also include isomers on double bond, ring, fused ring (E-, Z-, cis-, trans-isomer), isomers generated from asymmetric carbon atoms (R-, S-isomer, α-, β-configuration, enantiomer, diastereomer), optically active isomers (D-, L-, d-, l-isomer), polar compounds generated by chromatogaphic separation (more polar compound, less polar compound), equilibrium compounds, rotational isomers, mixtures thereof at any ratios and racemic mixtures.

Processes for the Preparation of the Compound of the Present Invention:

The compound represented by formula (I) may be prepared by methods described in U.S. Pat. No. 4,464,382, the compound represented by formula (II) may be prepared by methods described in JP-A-5-186472 and JP-A-5-345784, the compound represented by formula (III) may be prepared by methods described in U.S. Pat. No. 4,740,517 and the compound represented by formula (IV) may be prepared by methods described in U.S. Pat. No. 4,734,419 and U.S. Pat. No. 4,883,800.

Furthermore, the compound used in the present invention may be prepared by the method described in U.S. Pat. No. 4,883,800, EP218999, JP-A-60-89469, EP307879, EP189272, JP-A-2-72144, EP714893, WO99/50268, EP355827, EP355827, EP353198, U.S. Pat. No. 4,474,967, EP222576, U.S. Pat. No. 4,853,401, CA2143603, EP33617, EP243018, EP421365, U.S. Pat. No. 4,439,617, EP2895, EP365324, WO97/32863, EP492667, JP-A-7-10857, EP1236720, EP1236720, WO92/17446, JP-A-2002-241347, EP269355, JP-A-62-67075, EP252713, EP305947, EP322255, WO98/28265, EP17379, or WO89/09773.

Pharmacological Activities:

The compound in the present invention is efficacious in a gait disturbance model of cauda equina compression, known as a spiral canal stenosis model. Therefore, aldose reductase inhibitory compounds are useful for spinal canal stenosis, and can improve motor function, especially muscle weakness, muscle weakness, and decreasing in walking ability of spinal canal stenosis patients. Furthermore, the compound is useful for paralysis, hypoesthesia, pain, or numbness of patients, especially lower limb paralysis, hypoesthesia, pain or numbness. In addition, it is effective in the therapy for bladder or rectum disorder due to spinal canal stenosis. Bladder disorder due to spinal canal stenosis means dysuria due to it. It includes frequent miction, delayed urination, forceless urinary stream, ischuria and urinary incontinence. Furthermore, rectal disorder due to spinal canal stenosis means defecation disorder due to it.

The effect of therapy for spiral canal stenosis by the compound in the present invention is thought to be based on the improvement of hypofunction of the surrounding tissue of spinal canal, for example intervertebral disk, or the improvement of hyperplasia of yellow ligament, posterior ligament or the like, the improvement of inflammation or reduction of blood flow due to nerve compression, or the nerve protection.

Toxicity:

The toxicity of the compound of the present invention is very low, and it is confirmed that the compound is safe enough for pharmaceutical use. For example, in the case of oral administration of epalrestat to rat, LD₅₀ is 5600 mg/kg.

Application to Pharmaceuticals:

A combination agent obtained by combining the compound of formula (I) or a non-toxic salt thereof with other medicaments may be administered to accomplish the following purposes:

1) to supplement and/or enhance the preventive and/or therapeutic effect of the present compound;

2) to improve the kinetics and/or absorption and reduce the dose of the present compound; and/or

3) to eliminate the side effects of the present compound; and

A combination of the compound of the present invention and other medicaments may be administered in the form of the formulations having these components incorporated in one preparation, or may be administered in separate preparations. In the case where these medicaments are administered in separate preparations, they may be administered simultaneously or at different times. In the latter case, the compound of the present invention may be administered before the other medicaments. Alternatively, the other medicaments may be administered before the compound of the present invention. The method for the administration of these medicaments are the same or different.

The diseases on which the preventive and/or therapeutic effect of the above mentioned combination preparations works are not specifically limited but may be those for which the preventive and/or therapeutic effect of the compound of the present invention is supplemented and/or enhanced.

The other pharmaceutical for supplementing and/or enhancing the prevention and/or treatment effect of the compound of the present invention for spiral canal stenosis includes, for example prostaglandins, prostaglandin derivatives, nonsteroidal anti-inflammatory drug (NSAID), vitamin, muscle relaxant, anti-depressant, poly ADP-ribose polymerase (PARP) inhibitor, excitatory amino acid receptor antagonist (such as NMDA receptor antagonist and AMPA receptor antagonist), radical scavenger, astrocyte modulating agent, IL-8 receptor antagonist, immunosuppressive drug (such as FK506 and cyclosporine).

Examples of prostaglandins (hereinafter, abbreviated as PG) include PG receptor agonists, PG receptor antagonists, and the like.

Examples of PG receptors include PGE receptors (EP1, EP2, EP3 and EP4), PGD receptors (DP, CRTH2), PGF receptors (FP), PGI receptors (IP), TX receptors (TP), and the like. In addition, examples of prostaglandin derivative formulations include limaprost, limaprost alfadex, beraprost and the like.

Examples of NSAID include sasapyrine, sodium salicylate, aspirin, aspirin dialuminate, diflunisal, indomethacin, suprofen, ufenamate, dimethylisopropylazulene, bufexamac, felbinac, diclofenac, tolmetin sodium, clinoril, fenbufen, nabumetone, proglumetacin, indomethacin farnesyl, acemetacin, proglumetacin maleate, amfenac sodium, mofezolac, etodolac, ibuprofen, ibuprofen piconol, naproxen, flurbiprofen, flurbiprofen axetil, ketoprofen, Fenoprofen calcium salt, tiaprofenic acid, oxaprozin, pranoprofen, loxoprofen sodium, alminoprofen, zaltoprofen, mefenamic acid, mefenamic acid aluminium, tolfenamic acid, floctafenine, ketophenylbutazone, oxiphenbutazone, piroxicam, tenoxicam, ampiroxicam, napageln ointment, epirizole, tiaramide hydrochloride, tinoridine hydrochloride, emorfazone, sulpvrine, migrenin, Saridon, Sedes G, amipylo-N, solvon, pyrine compounding cold medicine, acetaminophen, phenacetin, dimetotiazine mesilate, cimetoride-combined drug, non-pyrine-combined cold medicine and the like.

Examples of muscle relaxant include tolperisone hydrochloride, chlorzoxazone, chlormezanone, methocarbamol, phenprobamate, pridinol mesilate, chlorphenesin carbamate, baclofen, eperisone hydrochloride, afloqualone, tizaindine hydrochloride, alcuronium chloride, suxamethonium chloride, tubocurarine chloride, dantrolene sodium, pancuronium bromide, vecuronium bromide and the like.

Examples of tricyclic antidepressant include imipramine hydrochloride, desipramine hydrochloride, clomipramine hydrochloride, trimipramine maleate, amitriptyline hydrochloride, nortriptyline hydrochloride, lofepramine hydrochloride, amoxapine, dosulepin hydrochloride and the like.

Examples of tetracyclic antidepressant include maprotiline, mianserin and the like.

The weight ratio of the compound of the present invention and the other medicaments is not specifically limited.

The other medicaments may be administered as a combination of same or different kind of more than two arbitrary medicants.

Furthermore, the other medicaments for supplementing and/or enhancing the preventive and/or therapeutic effect of the present invention include not only those found so far but also those which will be found on the basis of the above mentioned mechanism.

The compounds of the present invention naturally include all salts prepared by known methods. The pharmacologically acceptable salts are preferable. It is confirmed that all the compounds described in the Specification and the Claims have pharmacologically acceptable low-toxicity and safe enough for pharmaceutical use.

The pharmacologically acceptable salts described in the present invention are for example, alkaline metal, alkaline earth metal, ammonium salt or salt with amine when a parent compound is an acidic compound, and for example organic or inorganic acid addition salt when a parent compound is a basic compound.

In addition, the compound of the present invention may be administrated the following acid addition salt thereof. The acid addition salts are preferably non-toxic and water-soluble. The appropriate acid addition salts include, for example, salts of inorganic acids (hydrochloride, hydrobromide, hydroiodide, sulfate, phosphate, nitrate, etc.), salts of organic acids (acetate, trifluoroacetate, lactate, tartrate, oxalate, fumarate, maleate, benzoate, citrate, methanesulfonate, ethanesulfonate, benzenesulfonate, toluenesulfonate, isethionate, glucuronate, gluconate, etc.) and the like. Hydrochloride is preferable as an acid addition salt.

Furthermore, the compounds used in the present invention or salts thereof may be solvates thereof

The solvates are preferably non-toxic and water-soluble. The appropriate solvates include, for example, solvates such as water, alcohol solvents (ethanol, etc.), and the like.

In addition, the compounds used in the present invention may be prodrugs prepared by a known method.

The prodrug for the compound of the present invention means a compound which is converted into the compound of the present invention by reaction with an enzyme, a gastric acid, or the like, in the living body. Examples of the prodrug for the compound of the present invention include a compound wherein hydroxyl of the compound of the present invention is substituted with acyl, alkyl, phosphoric acid, boric acid, or the like (e.g., a compound wherein hydroxyl of the compound of the present invention is modified with acetyl, palmitoyl, propanoyl, pivaloyl, succinyl, fumaryl, alanyl, dimethylaminomethylcarbonyl, etc.); a compound wherein carboxyl of the compound of the present invention is modified with ester, amide, or the like (e.g., a compound wherein carboxyl of the compound of the present invention is modified with ethyl ester, phenyl ester, carboxymethyl ester, dimethyl aminomethyl ester, pivaloyloxymethyl ester, ethoxycarbonyloxyethyl ester, phthalidyl ester, (5-methyl-2-oxo-1,3-dioxolen-4-yl)methyl ester, cyclohexyloxycarbonylethyl ester, methyl amide, etc.); and the like. In addition, the prodrug for the compound of the present invention may hydrate or non-hydrate.

The compound of the present invention and ester thereof may be converted into α-, β-, or γ-cyclodextrin, or cyclodextrin clathrate compound using α-, β-, or γ-cyclodextrin by the method described in GB1351238 or GB1419221. It is convenient for the use of drug because it increases its stability and its water solubility by converting into cyclodextrin clathrate.

For the purpose above described, the compound used in the present invention, or a combination of the present invention compound and other medicaments may be normally administered systemically or locally, usually by oral or parenteral administration.

The doses to be administered are determined depending upon, for example, ages, body weights, symptoms, the desired therapeutic effects, the route of administration and the duration of the treatment. For the human adult, the doses per person are generally from 0.1 ng to 100 mg, by oral administration, up to several times per day, and from 0.1 ng to 10 mg, by parenteral administration (preferably intravenous injection), up to several times per day, or continuous administration 1 to 24 hours per day from vein.

As mentioned above, the doses to be used depend upon various conditions. Therefore, there are cases in which doses lower than or greater than the ranges specified above may be used.

To administer the compounds in the present invention, use is made of solid preparations for internal use and liquid preparations for internal use for oral administration as well as preparations for injections, external preparations, suppositories, eye drops, inhalations and the like for parenteral administration.

Examples of the solid preparations for internal use for oral administration include tablets, pills, capsules, powders, granules and the like. The capsules include hard capsules and soft capsules. The tablets include sublingual tablets, intraoral patches, orally fast disintegrating tablets and the like.

Such a solid preparation for internal use is prepared by a formulation method commonly employed by using one or two or more active substances either as it is or as a mixture with an excipient (lactose, mannitol, glucose, microcrystalline cellulose, starch, etc.), a binder (hydroxypropylcellulose, polyvinylpyrrolidone, magnesium metasilicate aluminate, etc.), a disintegrating agent (calcium cellulose glycolate, etc.), a lubricant (magnesium stearate, etc.), a stabilizer and a dissolution aid (glutamic acid, aspartic acid, etc.). If necessary, it may be coated with a coating agent (sucrose, gelatin, hydroxypropylcellulose, hydroxypropylmethylcellulose phthalate, etc.). It may be coated with two or more layers. Moreover, capsules made of an absorbable material such as gelatin are involved in the scope thereof.

The sublingual tablets may be prepared in accordance with a well known method. For example, a sublingual tablet is prepared by a formulation method commonly employed by using one or more active substances are used mixed with an excipient (lactose, mannitol, glucose, microcrystalline cellulose, starch, etc.), a binder (hydroxypropylcellulose, polyvinylpyrrolidone, magnesium metasilicate aluminate, etc.), a disintegrator (starch, L-hydroxypropyl cellulose, carboxymethyl cellulose, croscarmellose sodium, calcium cellulose glycolate, etc.), a lubricant (magnesium stearate, etc.), a swelling agent (hydroxypropyl cellulose, hydroxylpropylmethy cellulose, carbopol, carboxymethyl cellulose, polyvinyl alcohol, xanthan gum, guar gum, etc.), a swelling aid agent (glucose, fructose, mannitol, xylitol, erythritol, maltose, trehalose, phosphate, citrate, silicate, glycine, glutamic acid, arginine, etc.), a stabilizer and a dissolution aid (polyethylene glycol, propylene glycol, glutamic acid, aspartic acid, etc.), a flavoring agent (orange, strawberry, mint, lemon, vanilla, etc.). If necessary, it may be coated with a coating agent (sucrose, gelatin, hydroxypropylcellulose, hydroxypropylmethylcellulose phthalate, etc.). If necessary, it may be coated with two or more layers. Moreover, it may also further comprise some additives such as sweetening agents, antioxidants, coloring agents, preservatives and the like.

The intraoral patch may be prepared in accordance with a well known method. For example, a intraoral patch is prepared by a formulation method commonly employed by using one or more active substances are used mixed with an excipient (lactose, mannitol, glucose, microcrystalline cellulose, starch, etc.), a binder (hydroxypropylcellulose, polyvinylpyrrolidone, magnesium metasilicate aluminate, etc.), a disintegrator (starch, L-hydroxypropyl cellulose, carboxymethyl cellulose, croscarmellose sodium, calcium cellulose glycolate, etc.), a lubricant (magnesium stearate, etc.), a attach agent (hydroxypropyl cellulose, hydroxylpropylmethy cellulose, carbopol, carboxymethyl cellulose, polyvinyl alcohol, xanthan gum, guar gum, etc.), a attach aid agent (glucose, fructose, mannitol, xylitol, erythritol, maltose, trehalose, phosphate, citrate, silicate, glycine, glutamic acid, arginine, etc.), a stabilizer and a dissolution aid (polyethylene glycol, propylene glycol, glutamic acid, aspartic acid, etc.), a flavoring agent (orange, strawberry, mint, lemon, vanilla, etc.) and the like. If necessary, it may be coated with a coating agent (sucrose, gelatin, hydroxypropylcellulose, hydroxypropylmethylcellulose phthalate, etc.) and the like. If necessary, it may be coated with two or more layers. Moreover, it may also further comprise some additives such as sweetening agents, antioxidants, coloring agents, preservatives and the like.

The orally fast disintegrating tablets may be prepared in accordance with a well known method. For example, an orally fast disintegrating tablets is prepared by a formulation method commonly employed by using one or more active substances either as it is, or as a mixture bulk or granulated bulk materials which is coated with an adequate coating agent (ethyl cellulose, hydroxypropyl cellulose, hydroxylpropylmethy cellulose, acrylate-methacrylate-copolymer, etc.), a plasticizer (polyethylene glycol, triethyl citrate, etc.), with an excipient (lactose, mannitol, glucose, microcrystalline cellulose, starch, etc.), a binder (hydroxypropylcellulose, polyvinylpyrrolidone, magnesium metasilicate aluminate, etc.), a disintegrator (starch, L-hydroxypropyl cellulose, carboxymethyl cellulose, croscarmellose sodium, calcium cellulose glycolate, etc.), a lubricant (magnesium stearate, etc.), a dispersion aid (glucose, fructose, mannitol, xylitol, erythritol, maltose, trehalose, phosphate, citrate, silicate, glycine, glutamic acid, arginine, etc.), a stabilizer and a dissolution aid (polyethylene glycol, propylene glycol, glutamic acid, aspartic acid, etc.), a flavoring agent (orange, strawberry, mint, lemon, vanilla, etc.) and the like. If necessary, it may be coated with a coating agent (sucrose, gelatin, hydroxypropylcellulose, hydroxypropylmethylcellulose phthalate, etc.) and the like. If necessary, it may be coated with two or more layers. Moreover, it may also further comprise some additives such as sweetening agents, antioxidants, coloring agents, preservatives and the like.

The liquid preparations for internal use for oral administration include pharmaceutically acceptable aqueous solutions, suspensions, emulsions, syrups, elixirs and the like. Such a liquid preparation is prepared by dissolving, suspending or emulsifying one or more active substances in a diluent commonly employed (purified water, ethanol or a mixture thereof, etc.). Such liquid forms may also further comprise some additives such as humectants, suspending agents, emulsifying agents, sweetening agents, flavoring agents, aroma, preservatives, buffers and the like.

The dosage forms of the parenteral administration preparations for external use include ointments, gels, creams, fomentations, patches, liniments, atomized agents, inhalations, sprays, aerosols, eye drops, nasal drops and the like. Such a preparation contains one or more active substances and is prepared by a well known method or a commonly employed formulation.

Ointments are prepared in accordance with a well known formulation or a commonly employed formulation. For example, they are prepared by softening or melting one or two or more active substances in a base. The ointment base is selected from well known ones or those commonly employed. For example, use may be made of one base or a mixture of two or more thereof selected from higher fatty acids or higher fatty acid esters (adipic acid, myristic acid, palmitic acid, stearic acid, oleic acid, adipic acid esters, myristic acid esters, palmitic acid esters, stearic acid esters, oleic acid esters, etc.), waxes (beeswax, whale wax, ceresin, etc.), surfactants (polyoxyethylene alkyl ether phosphoric acid esters, etc.), higher alcohols (cetanol, stearyl alcohol, cetostearyl alcohol, etc.), silicone oils (dimethylpolysiloxane, etc.), hydrocarbons (hydrophilic vaseline, white vaseline, refined lanolin, liquid paraffin, etc.), glycols (ethylene glycol, diethylene glycol, propylene glycol, polyethylene glycol, macrogol, etc.), vegetable oils (castor oil, olive oil, sesame oil, turpentine oil, etc.), animal oils (mink oil, yolk oil, squalane, squalene, etc.), water, absorption promoters and skin irritation inhibitors. The ointments may further contain a humectant, a preservative, a stabilizer, an antioxidant, a flavor, and the like.

Gels are prepared in accordance with a well known formulation or a formulation commonly employed. For example, they are prepared by melting one or more active substances in a base. The gel base is selected from well known ones or those commonly employed. For example, use may be made of one base or a mixture of two or more thereof selected from lower alcohols (ethanol, isopropyl alcohol, etc.), gelling agents (carboxymethylcellulose, hydroxyethylcellulose, hydroxypropylcellulose, ethylcellulose, etc.), neutralizing agents (triethanolamine, diisopropanolamine, etc.), surfactants (polyethylene glycol monostearate, etc.), gums, water, absorption promoters and skin irritation inhibitors. The gels may further contain a preservative, an antioxidant, a flavor, and the like.

Creams are prepared in accordance with a well known formulation or a formulation commonly employed. For example, they are prepared by melting or emulsifying one or more active substances in a base. The cream base is selected from well known ones or those commonly employed. For example, use may be made of one base or a mixture of two or more thereof selected from higher fatty acid esters, lower alcohols, hydrocarbons, polyhydric alcohols (propylene glycol, 1,3-butylene glycol, etc.), higher alcohols (2-hexyldecanol, cetanol, etc.), emulsifiers (polyoxyethylene alkyl ethers, fatty acid esters, etc.), water, absorption promoters and skin irritation inhibitors. The creams may further contain a preservative, an antioxidant, a flavor, and the like.

Fomentations are prepared in accordance with a well known formulation or a formulation commonly employed. For example, they are prepared by melting one or more active substances in a base, kneading and then applying and spreading the kneaded matter on a substrate. The fomentation base is selected from well known ones or those commonly employed. For example, use may be made of one base or a mixture of two or more thereof selected from thickeners (polyacrylic acid, polyvinylpyrrolidone, gum acacia, starch, gelatin, methylcellulose, etc.), moistening agents (urea, glycerin, propylene glycol, etc.), fillers (kaolin, zinc oxide, talc, calcium, magnesium, etc.), water, dissolution aids, tackifiers and skin irritation inhibitors. The fomentations may further contain a preservative, an antioxidant, a flavor, and the like.

Patches are prepared in accordance with a well known formulation or a formulation commonly employed. For example, they are prepared by melting one or more active substances in a base and then applying and spreading on a substrate. The patch base is selected from well known ones or those commonly employed. For example, use may be made of one base or a mixture of two or more thereof selected from polymer bases, fats and oils, higher fatty acids, tackifiers and skin irritation inhibitors. The patches may further contain a preservative, an antioxidant, a flavor, and the like.

Liniments are prepared in accordance with a well known formulation or a formulation commonly employed. For example, they are prepared by dissolving, suspending or emulsifying one or two or more active substances in one or more media selected from water, alcohols (ethanol, polyethylene glycol, etc.), higher fatty acids, glycerin, soap, emulsifiers, suspending agents, and the like. The liniments may further contain a preservative, an antioxidant, a flavor, and the like.

Atomized agents, inhalations and sprays may contain, in addition, to a diluent commonly employed, a stabilizer such as sodium hydrogen sulfite, a buffering agent for imparting isotonicity, for example, an isotonic agent such as sodium chloride, sodium citrate or citric acid. Methods for producing a spray are described in detail in, for example, U.S. Pat. No. 2,868,691 and U.S. Pat. No. 3,095,355.

The injections for parenteral administration include solutions, suspensions, emulsions and solid injections to be dissolved or suspended before use. Such an injection is used by dissolving, suspending or emulsifying one or more active substances in a solvent. The solvent includes, for example, distilled water for injection, physiological saline, vegetable oils, alcohols such as propylene glycol, polyethylene glycol and ethanol, and mixtures thereof The injection may further contain a stabilizer, a dissolution aid (glutamic acid, aspartic acid, Polysorbate 80 (registered trademark), etc.), a suspending agent, an emulsifier, a soothing agent, a buffer, a preservative, and the like. Such an injection may be produced by sterilizing at the final step or employing an aseptic process. Alternatively, it is also possible that an aseptic solid product such as a freeze-dried product is produced and sterilized or dissolved in aseptic distilled water for injection or another solvent before use.

The inhalations for parenteral administration include aerosols, powders for inhalation and liquids for inhalation. Such inhalations may be dissolved or suspended in water or another adequate medium for use.

The inhalations may be prepared in accordance with a well known method.

For example, liquid preparations for inhalation may be, if necessary, prepared by appropriately selecting a preservative (benzalkonium chloride, paraben, etc.), a colorant, a buffering agent (sodium phosphate, sodium acetate, etc.), an isotonic agent (sodium chloride, concentrated glycerin, etc.), a thickener (carboxyvinyl polymer, etc.), an absorption promoter, and the like.

Powders for inhalation may be prepared, if necessary, by appropriately selecting a lubricant (stearic acid and its salt, etc.), a binder (starch, dextrin, etc.), an excipient (lactose, cellulose, etc.), a colorant, a preservative (benzalkonium chloride, paraben, etc.), an absorption promoter, and the like.

When the liquids for inhalation are administered, a sprayer (atomizer, nebulizer) is usually used. When the powders for inhalation are used, an inhalation administration apparatus for powder agents is usually used.

Other compositions for parenteral administration include suppositories and pessaries for vaginal administration which contain one or more active substances, and are prepared in accordance with common formulations.

Sustained-release drug may be supplied the compound of the present invention directly to affected area continuously. Administration types of sustained-release drug include implantation and the like.

Examples of biodegenerative polymer used as compositions for prolonged delivery of therapeutic agents of the present invention include aliphatic acid polyesters or copolymer thereof, polyacrylic acid esters, polyhydroxybutyric acids, polyalkylene oxate, polyorthoesters, polycarbonates, polyamides and the like. These compounds may be used singly or in admixture of two or more thereof Examples of the aliphatic acid ester polymers and copolymers thereof include polylactic acid, polyglycolic acid, polycitric acid, polymalic acid, poly-ε-caprolactone, polydioxanone and polyphosphazene. Examples of aliphatic acid polyesters or copolymer thereof include graft, block, alternation and random copolymer and two or more thereof. These compounds may be used singly or in admixture of two or more thereof. Besides these compounds, poly-α-cyanoacrylic acid esters, poly-β-hydroxybutyric acids, polytrimethyleneoxates, polyorthoesters, polyorthocarbonates, polyethylene carbonates, poly-γ-benzyl-L-glutamic acids, poly-L-alanines and copolymer of two or more above described materials may be used singly or in admixture of two or more thereof Preferred among these compounds are polylactic acids, polyglycolic acids and lactic acid-glycolic acid copolymers, more preferably lactic acid-glycolic acid copolymers.

Examples of lactic acid used as polylactic acid or lactic acid-glycolic acid copolymer include L-lactic acid, DL-lactic acid and the like.

The average molecular weight of these in vivo degradable polymers to be used in the invention is preferably from about 2,000 to 800,000, more preferably from about 5,000 to 200,000. For example, the polylactic acid preferably has a weight-average molecular weight of from about 5,000 to 100,000, more preferably from about 6,000 to 50,000. The polylactic acid can be synthesized according to any known preparation method per se.

In the lactic acid-glycolic acid copolymer, the composition ratio of the lactic acid to the glycolic acid is preferably from about 100/0 to 0/100 (w/w), particularly from about 90/10 to 30/70 (w/w). The weight-average molecular weight of the lactic acid-glycolic acid copolymer is preferably from about 5,000 to 100,000, more preferably from about 10,000 to 80,000. The lactic acid-glycolic acid copolymer can be synthesized according to any known preparation method per se.

### INDUSTRIAL APPLICABILITY

An aldose inhibitory compound is effective for prevention and/ or therapy for spinal canal stenosis and the like, such as cervical spinal canal stenosis, thoracic spinal canal stenosis, lumbar spinal canal stenosis and wide spinal canal stenosis and the like. Concretely, it has effect of improving motor action, especially reduction of muscle power, intermittent claudication and gait disability.

### BRIEF DESCRIPTION OF THE DRAWINGS

FIG. 1 shows that administration of Compound A (5-[(1Z,2E)-2-methylphenylpropenylidene]-4-oxo-2-thioxo-3-thiazolidineacetic acid) is improved the pathology in rats of gait disturbance model by cauda equina compression.

FIG. 2 shows that administration of Compound B ((R)-2-(4-bromo-2-fluorobenzyl)spiro[1,2,3,4-tetrahydropyrrolo[1,2-a]pyrazine-4,3′-pyrrolidine]-1,2′,3,5′-tetrone) or Compound C ((2S,4S)-6-fluoro-2′,5′-dioxospiro[3,4-dihydro-2H-1-benzopyran-4,4′-imidazoline]-2-carboxamide) is improved the pathology in rats of gait disturbance model by cauda equina compression.

### BEST MODE FOR CARRYING OUT THE INVENTION

The present invention is explained below in detail based on Examples and Formulation Examples, but the present invention is not limited thereto.

### EXAMPLE 1

Improvement effect of this invented compound in a rat model of gait disturbance model by cauda equina compression:

<Method of Making an Animal Model>

A rat of gait disturbance model by cauda equina compression was made by the method of Takenobu et al. (J. Neurosci. Methods, 104(2), 191-198 (2002)). Namely, a rat was anesthetized by sodium pentobarbital, removed its dorsal hair and then was fixed its body in the prone position. After disinfection of the back with Chlorhexidine gluconate (5% Hibiten Liquid: Sumitomo Pharmaceuticals), the lumbar was incised along the midline to expose the spine. After excision of the fifth lumbar spinous process, silicon rubber 1×4×1.25 mm (height×length×width) were inserted into the fourth and the sixth lumbar spinal canals from small holes of vertebral arch which was made by mini-drill. Benzylpenicillinpotassium (penicellin G potassium Meiji; Meiji Seika) was dropped into the incised part and injected into femor muscle. Muscle and skin of the incised part were closed by surgical suture. Sutured part was painted with iodine tincture.

A sham-operated rat was made by the above described method except for the insertion of silicon rubber.

<Examination of Walking Ability>

The walking ability was examined by using the treadmill apparatus.

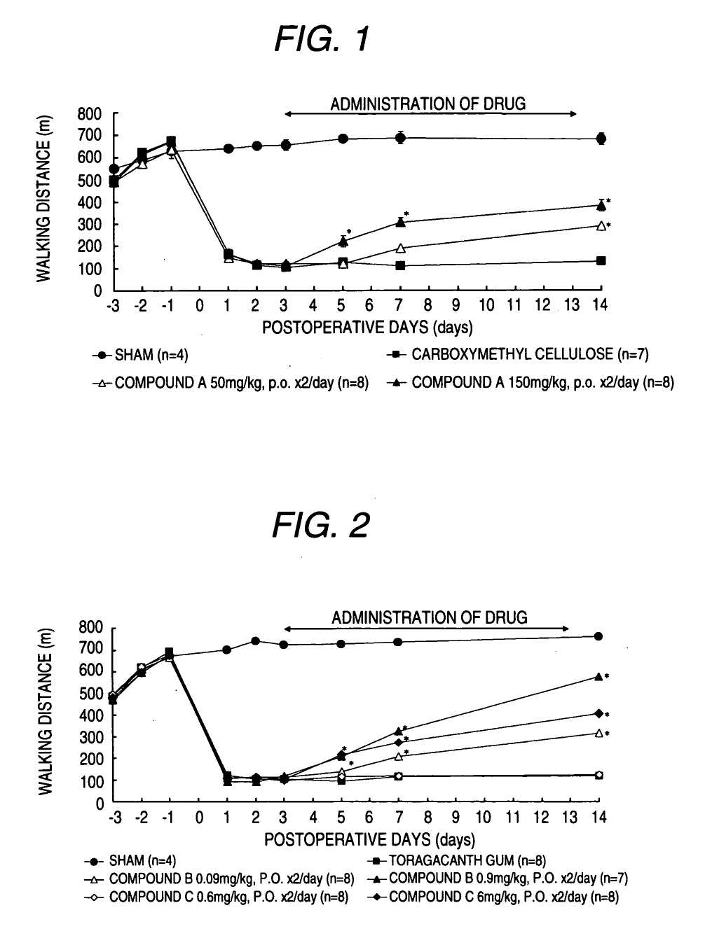

The rats were put on the running belt, and adapted to the condition where the grid was sent an electric current (0.04 mA-4 mA) for three minutes or more. Animals were forced to walk by an initial speed of 10 m/min, which was gradually increased by 5 m/min at 3 min intervals. Electric stimulation (0.04 mA-4 mA) was given to the rats that stopped walking and moved to the grid for electric stimulation equipped in front of the running belt. The distance between the point to walk and the point to give up walk, in other words, the point where it is impossible for them to walk even though the stimulation (sound, contact and electricity) to make them walk was given, was measured with the mileometer built into the equipment. The walking training was performed once a day for three consecutive days before the operation. After the operation, aldose reductase inhibitors, 5-[(1Z,2E)-2-methylphenylpropenylidene]-4-oxo-2-thioxo-3-thiazolidineacetic acid (in FIG. 1, Compound A; generic name: epalrestat), (R)-2-(4-bromo-2-fluorobenzyl)spiro[1,2,3,4-tetrahydropyrrolo[1,2-a]pyrazine-4,3′-pyrrolidine]-1,2′,3,5′-tetrone (in FIG. 2, Compound B; AS-3201), or (2S,4S)-6-fluoro-2′,5′-dioxospiro[3,4-dihydro-2H-1-benzopyran-4,4′-imidazoline]-2-carboxamide (in FIG. 2, Compound C; SNK-860; generic name: fidarestat), was administered orally for 11 days. On the other hand, carboxymethylcellulose or toragacanth gum was administered as a negative control. The results obtained from the compounds- and negative control-groups were analyzed by the Dunnett's multiple comparison test (*P<0.05). FIG. 1 and FIG. 2 show the results.

<Results>

The gait disturbance model by cauda equina compression is reported as a model for the spinal canal stenosis. The compounds (Compound A, B or C) used for this invention improved the walking dysfunction in the gait disturbance model by cauda equina compression as shown in FIG. 1 and FIG. 2. That is, it was suggested that the compounds with the inhibition of the aldose reductase used for this invention could be effective for the treatment of the spinal canal stenosis.

### FORMULATION EXAMPLE 1

The following components were admixed in a conventional method and punched out to obtain 100 tablets each containing 50 mg of the active ingredient.

| | |
|---|---|
| Epalrestat (Compound A) | 5.0 g |
| Carboxymethyl cellulose calcium (disintegrating agent) | 0.2 g |
| Magnesium stearate (lubricant) | 0.1 g |
| Microcrystalline cellulose | 4.7 g |

### FORMULATION EXAMPLE 2

The following components were admixed in a conventional method, and the solution was sterilized in a conventional method, placed at 5 ml into ampoules and freeze-dried in a conventional method to thereby obtain 100 ampoules each containing 20 mg of the active ingredient.

| | | |
|---|---|---|
| Epalrestat (Compound A) | 5.0 | g |
| Mannitol | 20 | g |
| Distilled water | 500 | ml |

1. A method for prevention and/or treatment of spinal canal stenosis, which comprises administering to a mammal in need thereof an effective amount of an aldose reductase inhibitory compound.

2. The method according to claim 1, wherein the spinal canal stenosis is cervical spinal canal stenosis, thoracic spinal canal stenosis, lumbar spinal canal stenosis or wide spinal canal stenosis.

3. The method according to claim 1, wherein the method is used for improving paralysis, hypoesthesia, pain or numbness.

4. The method according to claim 1, wherein the method is used for improving physical ability.

5. The method according to claim 4, wherein the method for improving physical ability is used for improving reduction of muscle power, intermittent claudication and gait disability.

6. The method according to claim 1, wherein the method is used for improving dysuria or dyschezia.

7. The method according to claim 1, wherein the aldose reductase inhibitory compound is represented by formula (I): 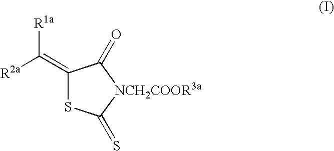
wherein
1) R^{1a} and R^{2a} are the same or different and each represents phenyl which may be substituted by at least one group selected from the following (1)-(10):
(1) halogen,
(2) trifluoromethyl,
(3) hydroxyl,
(4) nitro,
(5) carboxyl,
(6) amino which may be substituted by C1-4 alkyl,
(7) C1-4 alkyl, alkoxy or alkylthio,
(8) phenyl,
(9) a heterocyclic group containing at least one atom selected from a nitrogen atom, a sulfur atom and an oxygen atom, which may be substituted by at least one group selected from (a) halogen, (b) trifluoromethyl, (c) phenyl, (d) nitro, (e) hydroxyl, (f) carboxyl, (g) amino which may be substituted by C1-4 alkyl, (h) C1-4 alkyl, (j) C1-4 alkoxy, and (k) C1-4 alkyl
(10) C1-4 alkyl substituted by at least one substituent selected from hydroxyl, phenyl, and the heterocyclic group described above (9),
2) R^{1a} is hydrogen and R^{2a} is the following (1)-(6):
(1) C4-7 cycloalkyl or cycloalkenyl which may be substituted by at least one C1-4 alkyl,
(2) anthryl or naphthyl,
(3) phenyl which may be substituted by at least one group selected from the following (a)-(k):
(a) halogen,
(b) trifluoromethyl,
(c) hydroxyl,
(d) nitro,
(e) carboxyl,
(f) amino which may be substituted by C1-4 alkyl,
(g) C1-4 alkyl, alkoxy or alkylthio,
(h) phenyl,
(j) a heterocyclic group containing at least one atom selected from a nitrogen atom, a sulfur atom and an oxygen atom, which may be substituted by at least one group selected from (i) halogen, (ii) trifluoromethyl, (iii) phenyl, (iv) nitro, (v) hydroxyl, (vi) carboxyl, (vii) amino which may be substituted by C1-4 alkyl, (viii) C1-4 alkyl, (ix) C1-4 alkoxy, and (x) C1-4 alkylthio,
(k) C1-4 alkyl substituted by at least one substituent selected from hydroxyl, phenyl, and the heterocyclic group described above (j),
(4) a heterocyclic group containing at least one atom selected from a nitrogen atom, a sulfur atom and an oxygen atom, which may be substituted by at least one group selected from the following (a)-(k):
(a) halogen,
(b) trifluoromethyl,
(c) phenyl,
(d) nitro,
(e) hydroxyl,
(f) carboxyl,
(g) amino which may be substituted by C1-4 alkyl,
(h) C1-4 alkyl, alkoxy or alkylthio,
(j) oxo, or
(k) C1-4 alkyl substituted by at least one substituent selected from hydroxyl, phenyl, and the heterocyclic group described above (j) in (3), 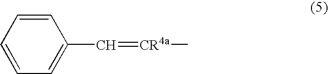
wherein R^{4a} is hydrogen or C1-4 alkyl, or 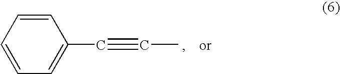
3) R^{1a} taken together with R^{2a} is tetramethylene or pentamethlene;
R^{3a} is
(1) hydrogen,
(2) C1-12 alkyl,
(3) C7-13 aralkyl,
(4) C4-7 cycloalkyl or cycloalkenyl which may be substituted by at least one C1-4 alkyl,
(5) phenyl which may be substituted by at least one group selected from the following (a)-(k):
(a) halogen,
(b) trifluoromethyl,
(c) hydroxyl,
(d) nitro,
(e) carboxyl,
(f) amino which may be substituted by C1-4 alkyl,
(g) alkoxy or alkythio which may be substituted by C1-4 alkyl,
(h) phenyl,
(j) a heterocyclic group containing at least one atom selected from a nitrogen atom, a sulfur atom and an oxygen atom, which may be substituted by at least one group selected from (i) halogen, (ii) trifluoromethyl, (iii) phenyl, (iv) nitro, (v) hydroxyl, (vi) carboxyl, (vii) amino which may be substituted by C1-4 alkyl, (viii) C1-4 alkyl, (ix) C1-4 alkoxy, and (x) C1-4 alkylthio, and,
(k) C1-4 alkyl substituted by at least one substituent selected from hydroxyl, phenyl, and the heterocyclic group described above (j), or
a salt of acid thereof when R^{3a} represents hydrogen, or a solvate thereof.

8. The method according to claim 1, wherein the aldose reductase inhibitory compound is a compound represented by formula (II): 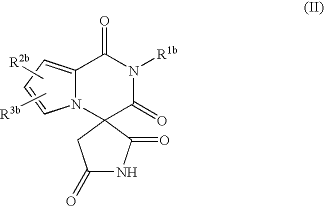
wherein R^{1b} represents (1) hydrogen, (2) lower alkyl, (3) substituted or unsubstituted aryl(lower alkyl), (4) substituted or unsubstituted aryl, or (5) 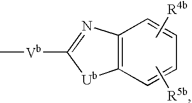
wherein R^{4b} and R^{5b} are the same or different and each represents (a) hydrogen, (b) halogen, (c) trifluoromethyl, (d) lower alkyl, (e) lower alkoxy, (f) acyl, (g) nitro, (h) amino, (i) lower alkylamino, or (j) di(lower alkyl)amino; U^{b} represents (a) oxygen, (b) sulfur, or (c) —NR^{6b}—wherein NR^{6b} represents hydrogen or lower alkyl, and V^{b} represents lower alkyl;
wherein R^{2b} and R^{3b} are the same or different and each represents (1) hydrogen, (2) halogen, (3) lower alkyl, (4) lower alkoxy, (5) acyl, (6) nitro, (7) amino, (8) lower alkylamino, (9) di(lower alkyl)amino, (10) allyl or (11) allyl which is substituted by lower alkyl, lower alkoxy or acyl, or
a salt thereof or a solvate thereof;
a compound represented by formula (III): 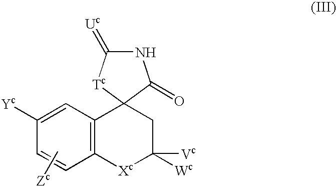
wherein T^{c} represents sulfur or NH;
U^{c} represents oxygen, sulfur or imino;
one of V^{c} and W^{c} represents hydrogen; halogenomethyl; 1H-tetrozol-5-yl; —COOR^{c} wherein R^{c} is hydrogen, alkyl, —(CH₂CH₂O)ₙCH₃ wherein n is an integer of 1 to 113, or substituted phenyl; 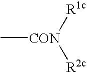
wherein R^{1c} and R^{2c} are the same or different and each represents hydrogen, alkyl, —(CH₂CH₂O)ₙCH₃ wherein n is an integer of 1 to 113; or substituted phenyl; —CH₂OR^{3c} wherein R^{3c} is hydrogen or alkyl; 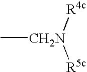
wherein R^{4c} and R^{5c} are the same or different and each represents hydrogen or alkyl, and the other represents hydrogen or alkyl;
X^{c} represents oxygen or sulfur;
Y^{c} and Z^{c} are the same or different and each represents hydrogen, halogen, alkyl, alkoxy, or alkylthio, or
a salt thereof or a solvate thereof; or
a compound represented by formula (IV): 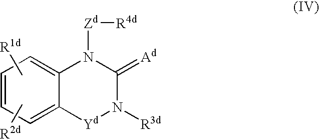
wherein R^{1d} and R^{2d} are the same or different and each represents hydrogen, halogen, lower alkoxy, or halo(lower alkyl);
R^{3d} represents (1) aryl or aryl(lower alkyl) which may be substituted, or (2) heterocyclic ring-(lower alkyl);
R^{4d} represents carboxy or protected carboxy;
A^{d} represents oxygen or sulfur;
Y^{d} represents carbonyl, thiocarbonyl, or sulfonyl;
Z^{d} represents lower alkylene, or
a salt thereof or a solvate thereof.

9. The method according to claim 7, wherein the aldose reductase inhibitory compound is 5-[(1Z,2E)-2-methylphenylpropenylidene]-4-oxo-2-thioxo-3-thiazolidine acetic acid.

10. The method according to claim 8, wherein the aldose reductase inhibitory compound is (R)-2-(4-bromo-2-fluorobenzyl)spiro[1,2,3,4-tetrahydropyrrolo[1,2-a]pyrazine-4,3′-pyrrolidine]-1,2′,3,5′-tetrone, (2S,4S)-6-fluoro-2′,5′-dioxospiro[3,4-dihydro-2H-1-benzopyran-4,4′-imidazolidine]-2-carboxamide or 2-[3-(4-bromo-2-fluorobenzyl)-7-chrolo-2,4-dioxo-1,2,3,4-tetrahydroquinazolin-1-yl]acetic acid.

11. The method according to claim 1, wherein the aldose reductase inhibitory compound is
(1) DL-spiro(2-fluoro-9H-fluoren-9,4′-imidazolidine)-2′,5′-dione,
(2) 2,7-difluoro-4,5-dimethoxyspiro[9H-fluoren-9,4′-imidazolidine]-2′,5′-dione,
(3) N-[3,5-dimethyl-4-(nitromethylsulphonyl)phenyl]-2-(2-methylphenyl)acetamide,
(4) N-(carboxymethyl)-7-fluoro-N-methyl-9-oxoxanthin-2-sulphoamide,
(5) 3-(4-methoxy-5-oxo-3-phenyl-2,5-dihydrofuran-2-yl)propanoic acid ethyl ester,
(6) 2-formamide-3-[5′-(2-formamide-1-hydroxyethyl)-2,2′-dihydroxybiphenyl-5-yl]-3-hydroxypropyonic acid,
(7) 2-[3-methyl-5-(4,5,7-trifluorobenzothiazol-2-ylmethyl)-phenyl]acetic acid,
(8) 2-[5-fluoro-2-[N-(3-nitrobenzyl)thiocarbamoyl]phenoxyacetic acid,
(9) 8′-chrolo-2′,3′-dihydrospiro[pyrrolidine-(3,6′)(5′H)-pyrro[1,2,3-de][1,4]benzoxazine]-2,5,5′-trione,
(10) 2-[1-(3,4-dichrolobenzyl)-2,4-dioxo-1,2,3,4-tetrahydroquinazolin-3-yl]acetic acid,
(11) 2-[4-(4,5,7-trifluorenzothiazol-2-yl)methyl-3-oxo-3,4-dihydro-2H-1,4-benzothiazin-2-yl]acetic acid,
(12) 1-(benzo[b]thiophen-2-ylsulphonyl)hydantoin,
(13) 1-(3-bromobenzo[b]furan-2-ylsulphonyl)hydanthione,
(14) 3-(carboxymethyl)-1-(3-nitrobenzyl)parabanic acid,
(15) 1′,3′-bis(acetoxymethyl)spiro[fluoren-9,4′-imidazolidine]-2′,5′-dione,
(16) 2,8-diisopropyl-3-thioxo-3,4-dihydro-2H-1,4-benzoxazine-4-acetic acid,
(17) N-[5-(trifluoromethyl)-6-methoxy-1-naphthalenyl]thioxomethyl]-N-methylglycine),
(18) (2,6-dimethylphenylsulphonyl)nitromethane,
(19) N-[4-(2,4-dioxothiazolidin-5-ylmethyl)phenyl]-1-phenyl-cyclopropane-1-carboxamide,
(20) 2-[3-oxo-4-(4,5,7-trifluorobenzothiazol-2-ylmethyl)-3,4-dihydro-2H-1,4-benzothiazin-2-yl]acetic acid,
(21) 2-[3,7-dimethylocta-2(E),6-didienyl]-2,3-epoxy-5,7-dihydroxy-6-methyl-1,2,3,4-tetrahydronaphthalene-1,4-dione,
(22) 6-fluoro-2-methylspiro[chroman-4,4′-imidazolidin]2′,5′-dione,
(23) (S)-6-fluorospiro(chroman-4,4′-imidazolidine)-2′,5′-dione,
(24) 3,4-dihydro-4-oxo-3-[[5-((trifluoromethyl)-2-benzothiazolyl]methyl]-1-phthalazine acetic acid,
(25) 5-(3-ethoxy-4-pentyloxyphenyl)-2,4-thiazolidinedione,
(26) 3-[(4-bromo-2-fluoro-phenyl)methyl]-3,4-dihydro-4-oxo-1-phthalazine acetic acid,
(27) ascorbyl gamolenate,
(28) ICI-10552,
(29) ICI-215918,
(30) JTT-811,
(31) lindolrestat,
(32) salfredins,
(33) TJN-732,
(34) TAT,
(35) thiazocin-A,
(36) axillarin, or
(37) minalrestat.

12. A medicine which comprises an aldose reductase inhibitory compound in combination with at least one pharmaceutical agent selected from prostaglandins, prostaglandin derivatives formulations, nonsteroidal anti-inflammatory drugs, vitamin compounds, muscle relaxants, antidepressants, poly ADP-ribose polymerase inhibitors, excitatory amino acid receptor antagonists, radical scavengers, astrocyte modulators, IL-8 receptor antagonists, and immunosuppressive drugs.

13-14. (canceled)

15. The method according to claim 1, wherein at least one pharmaceutical agent selected from prostaglandins, prostaglandin derivatives formulations, nonsteroidal anti-inflammatory drugs, vitamin compounds, muscle relaxants, antidepressants, poly ADP-ribose polymerase inhibitors, excitatory amino acid receptor antagonists, radical scavengers, astrocyte modulators, IL-8 receptor antagonists, and immunosuppressive drugs is administered in combination with the aldose reductase inhibitory compound.
